(19) United States
(12) Patent Application Publication
(10) Pub. No.: US 2007/0232929 A1
(43) Pub. Date: Oct. 4, 2007

(54) Skin detection sensor

(76) Inventors: George A. Kilgore, Colorado Springs, CO (US); Rand P. Whillock, North Oaks, MN (US); Gary E. Determan, Maple Grove, MN (US)

Correspondence Address: Kris T. Fredrick;Patent Services, Honeywell International Inc., 101 Columbia Road, Morristown, NJ, 07962, US

(21) Appl. No.: 11/726,556

(22) Filed: Mar. 22, 2007

Related U.S. Application Data
(63) Continuation-in-part of application No. 11/264,654, filed on Oct. 31, 2005.

(51) Int. Cl. A61B 6/00

(52) U.S. Cl. 600/473

(57) ABSTRACT

A device for detecting the presence of human skin including an illuminator source for providing an IR band capable of being absorbed by skin. A detector receives the IR band after the band has contacted the skin and provides a signal indicating the presence or absence of the IR band energy. A processing unit processes the signal and indicates whether skin has been presented when the band is substantially reflected, indicating no skin, and when it is substantially absent, indicating skin present. The preferred IR band wavelength is about 1.4 to about 2.2 μm.

### FIELD OF THE INVENTION

The present invention relates to skin detection sensors. More particularly, the invention relates to such sensors where light absorbing properties of skin are employed to detect the presence of human skin.

### BACKGROUND OF THE INVENTION

This invention is a continuation-in-part of co-pending U.S. patent application Ser. No. 11/264,654, filed Oct. 31, 2005, entitled “Skin Detection Sensor,” the disclosure of which is incorporated herein in its entirety. In that application, two IR bands are directed at a target proposed to be human skin. One IR band is capable of being reflected from skin and the other is absorbed by skin. When both conditions exist, skin is detected as being present.

The invention may be related to U.S. patent application Ser. No. 10/987,806, filed Nov. 12, 2004, and entitled “Infrared and Visible Fusion Face Recognition System”. The invention may also be related to U.S. patent application Ser. No. 10/946,748, filed Nov. 12, 2004, and entitled “Infrared Face Detection and Recognition System”. These above-mentioned patent applications are incorporated herein by reference. The assignee of these patent applications is the same assignee of the present invention.

Other related patent documents may include U.S. Pat. No. 6,370,260 B1, issued Apr. 9, 2002, entitled “Near-IR Human Detector”, and by Pavlidis et al.; U.S. Pat. No. 6,718,049 B2, issued Apr. 6, 2004, entitled “Near-Infrared Disguise Detection”, and by Pavlidis et al.; and U.S. patent application Ser. No. 10/077,672, filed Feb. 15, 2002, entitled “Near-Infrared Method and System for Use in Face Detection”, and by Pavlidis et al.; all of which are Incorporated herein by reference. The assignee of these patent documents is the same assignee of the present invention.

Current biometric sensors based on measuring fingerprint identity or hand geometry sensors are of significant interest to a variety of industries and applications. The security industry is constantly seeking sensors that identify certain specific persons to allow access to a secured area, a device such as a computer or other electronic equipment, or a cell phone.

However, biometric sensors that rely on a particular property of a person, such as a fingerprint, can be defeated. If a rubber cast or cutout of a fingerprint is used, the device will recognize the correct print, even if it is not being used by the person to whom it belongs. Photographs of irises have been used to thwart iris detection. Thus, access to secure property would be compromised.

Current biometric sensors may employ techniques to verify that a live person's finger is being presented to the sensor. These include techniques to measure skin conductivity or temperature. Techniques such as these can readily be defeated by simple countermeasures. It would be a great advance in the art if a device could be provided that would accurately identify the presence of human skin in the presence of determined countermeasures where an individual's specific and unique characteristic could then be recognized.

It would be another advantage in the art if the presence of human skin could be determined before a biometric sensor determines the existence of a specific person.

Yet another advantage would be if a device could be provided that would be simple and inexpensive to manufacture, small in size for incorporation into existing biometric sensors, and easy to use to permit or deny access to the biometric sensor.

Other advantages will appear hereinafter.

### SUMMARY OF THE INVENTION

It has now been discovered that the above and other advantages of the present invention may be obtained in the following manner. Specifically, the present invention provides a device for detecting the presence or absence of human skin.

The device includes an illuminator source for providing an IR band which contributes to the detection scheme. The IR band is capable of being absorbed by skin and thus, if skin is present, none or hardly any of the band is reflected. The preferred IR band wavelength is about 1.4 to about 2.2 μm.

The band of this invention could be transmitted at the same time as other bands not of interest in this invention.

A detector is positioned to receive the IR band after it has contacted the skin. The detector provides a signal indicating the presence or absence of the IR band. A processing unit processes the signal and indicates the presence of skin when the IR band is absent.

An electronic detection evaluator performs a weighted difference and threshold of the outputs of the detector. If the output voltage is pulled high, this indicates that the IR band absorbed, and thus human skin is present. If the weighted difference is below the threshold, the output is low, indicating reflection of the IR band and thus no human skin is present. This occurs when the IR band reflects off the skin.

### BRIEF DESCRIPTION OF THE DRAWINGS

For a more complete understanding of the invention, reference is hereby made to the drawings, in which:

FIG. 1 is a schematic view of the preferred embodiment of the present invention.

### DETAILED DESCRIPTION OF THE PREFERRED EMBODIMENT

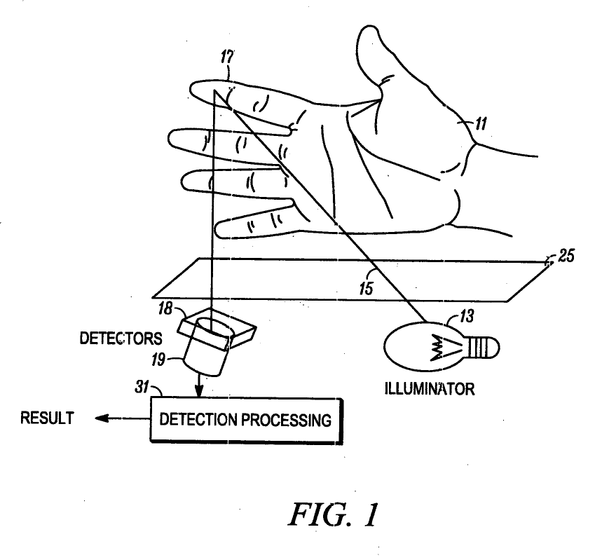

The present invention operates as a sensor to detect the presence of skin, such as that of the human hand or other part of the anatomy that is exposed to detection. In the preferred embodiment, a hand 11, as shown in FIG. 1, is illuminated by illuminator 13, which transmits infrared radiation (IR) at various selected wavelengths. These wavelengths are transmitted on a path that strikes the skin of hand 11 and is reflected back to one or more IR detectors.

The IR band 15 transmitted from illuminator 13 is in the band range that is absorbed by skin, and preferably of about 1.4 to about 2.2 μm. It has been discovered that this wavelength band range is absorbed almost completely by human skin 11. The path of the IR band from the finger 17 is received by detector 19. Detector 19 preferably includes filter 18 which passes only the band range of about 1.4 to about 2.2 μm and filters out other IR band wavelengths. Of course the illuminator can transmit a wider IR band than these preferred values.

A detection processing element 31, which may be a chip or other microelectronic device analyses the signal from the outputs of detector 19. If the signal is below a set threshold, the device output voltage is pulled low, indicating human skin is present. In this situation, a signal is almost zero since this band has been absorbed by skin 11.

If the output of the device 31 is high, indicating no human skin is present. This occurs when the IR band 15 reflects off the object it strikes, such as if a rubber or latex glove were worn over the skin and first detector will receive a signal indicating that no IR band has not been absorbed, as if something covered the human skin and second detector 19 will receive a lot of signal. The sensor will indicate that something other than human skin has been sensed. The detector processing element 31 may also be configured to use negative logic, such that there may be a negative output for detection of skin and a high output for the absence of skin. In any form, detector processing element 31 signals a positive result for skin when the expected absorbed band is either present or absent.

The present invention is admirably suited to improve many of the current biometric sensors currently in use or envisioned for use as a security device, access control or other use. Fingerprint sensors are able to compare a presented fingerprint against a data base, to allow or deny access to a controlled area or use of an electronic device such as a computer, cell phone, or other device, for example. But fingerprint sensors are not capable of distinguishing between a human finger presented for fingerprint screening and a rubber or plastic mold of the same finger. The present invention would verify the presence or absence of human skin, and thus make the biometric fingerprint sensor much more resistant to counermeasures. Iris comparisons also can be defeated by the use of contact lenses in some cases. Again, the present invention would verify the presence of actual skin, though obviously not transmitting directly into the eye. In one use of the present invention, the biometric sensor would need to have a positive determination of the presence of human skin from the device of this invention before even processing the data it has been designed to detect.

While particular embodiments of the present invention have been illustrated and described, it is not intended to limit the invention, except as defined by the following claims.

1. A device for detecting the presence of skin, comprising:
an illuminator source for providing an IR band being capable of being absorbed by skin;
a detector for receiving said IR band after said bands have contacted said skin and for providing a signal indicating the IR band's reflection or absorption; and
a detector processing unit for processing said signal and indicating the presence of skin when said IR band is substantially absent.

2. The device of claim 1, wherein said IR band is about 1.4 to about 2.2 μm.

3. The device of claim 1, wherein said illuminator is adapted to transmit an IR band that includes other IR band wavelengths.

4. The device of claim 3, wherein said detector processing unit is adapted to provide a signal indicating when said IR band is reflected to said detector.

5. The device of claim 1, wherein said detector further includes a filter that passes only said IR band.

6. A device for detecting the presence of skin, comprising:
illuminator source means for providing an IR band being capable of being absorbed by skin;
detector means for receiving said IR band after said bands have contacted said skin and for providing a signal indicating the IR band's reflection or absorption; and
detector processing unit means for processing said signal and indicating the presence of skin when said IR band is substantially absent.

7. The device of claim 6, wherein said IR band is about 1.4 to about 2.2 μm.

8. The device of claim 6, wherein said illuminator means is adapted to transmit an IR band that includes other IR band wavelengths.

9. The device of claim 8, wherein said detector processing unit means is adapted to provide a signal indicating when said IR band is reflected to said detector means.

10. The device of claim 6, wherein said detector means further includes a filter means for passing only said IR band.

11. A method for detecting the presence of skin, comprising:
providing an IR band being capable of being absorbed by skin an illuminator source;
receiving said IR band after said bands have contacted said skin on a detector for providing a signal indicating the IR band's reflection or absorption; and
processing said signal with a detector processing unit for and indicating the presence of skin when said IR band is substantially absent.

12. The method of claim 11, wherein said IR band is about 1.4 to about 2.2 μm.

13. The method of claim 11, wherein said illuminator is adapted to transmit an IR band that includes other IR band wavelengths.

14. The method of claim 13, wherein said detector processing unit is adapted to provide a signal indicating when said IR band is reflected to said detector.

15. The method of claim 11, which further contains the step of filtering with a filter that passes only said IR band prior to said band contacts said detector.
